(19) United States
(12) Patent Application Publication
(10) Pub. No.: US 2023/0058134 A1
(43) Pub. Date: Feb. 23, 2023

(54) ALDOSE REDUCTASE INHIBITORS FOR THE TREATMENT OF ACUTE RESPIRATORY DISTRESS SYNDROME, ACUTE LUNG INFLAMMATION/INJURY, CARDIAC INJURY AND ANTI-VIRAL THERAPY

(71) Applicant: Applied Therapeutics, Inc., New York, NY (US)

(72) Inventor: Shoshana SHENDELMAN, New York, NY (US)

(21) Appl. No.: 17/951,342

(22) Filed: Sep. 23, 2022

Related U.S. Application Data
(63) Continuation of application No. PCT/US2021/024876, filed on Mar. 30, 2021.
(60) Provisional application No. 63/002,538, filed on Mar. 31, 2020.
(60) Provisional application No. 63/006,264, filed on Apr. 7, 2020.

(51) Int. Cl. A61K 31/5025; A61P 31/14; A61P 11/00; A61P 9/04

(52) U.S. Cl. CPC A61K 31/5025; A61P 31/14; A61P 11/00; A61P 9/04

(57) ABSTRACT

The disclosure relates to methods for treating acute respiratory distress syndrome (ARDS), acute lung inflammation (ALI), acute lung injury and/or cardiac injury (e.g., acute cardiac injury), treating an infection, reducing pathogen burden and/or inhibiting pathogen replication by administering to a subject in need thereof a therapeutically effective amount of an aldose reductase inhibitor. In some aspects, the subject is infected with a respiratory pathogen and has influenza, SARS, MERS or COVID-19.

### RELATED APPLICATIONS

This application claims the benefit of U.S. Provisional Patent Application No. 63/002,538, filed on Mar. 31, 2020, and of U.S. Provisional Patent Application No. 63/006,264, filed on Apr. 7, 2020, the entire teachings of each of which are incorporated herein by reference.

### BACKGROUND

Aldose Reductase (AR) is an enzyme that plays a critical role in oxidative damage and inflammatory processes, and is recognized as being involved in diabetic complications, cardiovascular disease and a number of inflammatory diseases, such as atherosclerosis, asthma, and sepsis. AR is the rate-limiting enzyme in the polyol pathway, and the substrates for AR include glucose and other saturated and unsaturated aldehydes. AR catalytic activity requires NADPH. NADPH is also required for glutathione reductase activity, which is a major antioxidant mechanism in many cells. Thus, AR can compete with glutathione reductase for NADPH, resulting in redox imbalance and oxidative stress. AR has been shown to increase oxidative stress in hyperglycemeic and ischemic states. (Ravindranath et al Novel Role for Aldose Reductase in Mediating Acute Inflammatory Responses in the Lung, J Immunology 2009; 183:8128-8137.) Cardiopulmonary examples of AR mediated oxidative damage include acute lung inflammation (ALI), acute lung injury and cardiomyopathy.

An exaggerated inflammatory response following infection or injury in the lungs and the resultant increases in alveolar-capillary permeability and edema formation underlie the pathogenesis of acute respiratory distress syndrome (ARDS) and ALI. Sepsis-induce lung injury was shown to increase AR activity in the lungs in a mouse model. The increased AR activity in the lungs then led to increased IL-6-mediated inflammatory response, with increased cytokines, neutrophil infiltration into the lungs, and inflammatory activation of lung endothelial cells. Inhibition of AR activity (with small molecule inhibitors of AR) reduced or prevented all measures of the inflammatory response in the mouse model. AR was hypothesized to mediate this type of ALI through production of reactive oxygen species (ROS), which are known to activate the JNK/IL-6 inflammatory pathway. (Ravindranath et al Novel Role for Aldose Reductase in Mediating Acute Inflammatory Responses in the Lung, J Immunology 2009; 183:8128-8137)

Depletion of angiotensin converting enzyme 2 (ACE2) has been shown to result in cardiomyopathy and acute myocarditis in animal models. The mechanism by which this damage occurs is through increased oxidative stress, neutrophilic infiltration, inflammatory cytokine levels and collagenase levels. (Oudit et al Angiotensin II-mediated Oxidative Stress and Inflammation Mediate the Age-Dependent Cardiomyopathy in ACE2 Null Mice; Cardiovascular Research 75 (2007) 29-39.) Additionally, oxidative stress in cardiomyocytes is known to cause damage via the enzyme AR.

Many infectious agents, such as influenza viruses and coronaviruses, can affect the cardiovascular system and can damage the heart and/or lungs through inflammatory pathways, for example by causing myocarditis, cardiomyopathy and acute myocardial damage, and/or acute lung inflammation, which can lead to acute respiratory distress syndrome (ARDS) and acute lung injury. Severe acute respiratory syndrome coronavirus 2 (SARS-CoV-2), which causes coronavirus disease 2019 (COVID-19) reached a pandemic level in 2020. SARS-CoV-2 infection is mediated by binding of the viral spike protein to ACE2, which is highly expressed in the heart and lungs (Zhou P, Yang X, Wang X, et al. A pneumonia outbreak associated with a new coronavirus of probable bat origin. Nature. 2020; 579:270-273. doi.org/10.1038/s41586-020-2012-7) SARS-CoV-2/COVID-19 can cause significant cardiac morbidities, including cardiomyopathy and arrhythmia. Cardiac injury has been observed in severe COVID-19 cases and is strongly associated with mortality. ARDS is also strongly associated with COVID-19 mortality and also with influenza mortality. (Madjid M, Safavi-Naeini P, Solomon SD, Vardeny O. Potential Effects of Coronaviruses on the Cardiovascular System: A Review. JAMA Cardiol. Published online Mar. 27, 2020. doi:10.1001/jamacardio.2020.1286; Kalil, A. C., Thomas, P. G. Influenza virus-related critical illness: pathophysiology and epidemiology. Crit Care 23, 258 (2019). doi.org/10.1186/s13054-019-2539-x)

Some respiratory pathogens infect hosts (e.g., humans) through the upper and lower respiratory tract, and produce severe disease when the pathogen quickly replicates and establishes infection characterized by a high pathogen burden and/or infection in the lower respiratory tract. It is recognized that the replication rate of some viruses and viral burden is increased by host responses to infection, in particular the host inflammatory response. (See, e.g., Mocarski E S Jr. Virus self-improvement through inflammation: no pain, no gain. Proc Natl Acad Sci USA. 2002;99(6):3362-3364 doi:10.1073/pnas.072075899; Agueiler E R and Lenz L L. Inflammation as a Modulattor of Host Susceptibility to Pulmonary Influenza, Pneumococcal, and Co-Infections. Front. Immunol. 11:105. doi:10.3389//fimmu.2020.00105.)

There is a need for new therapeutic approaches to treat infections such as respiratory infections, including infections with viral pathogens such as influenza and coronaviruses (e.g., SARS-CoV-1, MERS-CoV-1, SARS-CoV-2 (COVID-19). There is a need for new therapeutic approaches to treat ALI, ARDS and cardiac injury, including infection-related ALI, ARDS and cardiac injury.

### SUMMARY

This disclosure relates to the use of an aldose reductase inhibitor (ARI) for the treatment of infection, reducing pathogen burden and/or inhibiting pathogen replication, in a subject that has an infection. In some aspects the pathogen is a virus, and in particular a respiratory virus, such as influenza virus or coronavirus (e.g., SARS-CoV-1, SARS-CoV-2, MERS-CoV).

This disclosure relates to the use of an ARI for the treatment of acute respiratory distress syndrome (ARDS), acute lung inflammation (ALI) and/or acute lung injury, including ARDS, ALI and acute lung injury in a subject that has an infection and wherein the ARDS, ALI and/or acute lung injury is related to are caused by the infection (e.g., COVID-19). This disclosure also relates to the use of an ARI for the treatment of cardiac injury (e.g., acute cardiac injury), for example cardiac injury that is related to our caused by an infection (e.g., COVID-19).

This disclosure relates to an aldose reductase inhibitor (ARI) for use in treating acute respiratory distress syndrome (ARDS), and a method for treating acute respiratory distress syndrome (ARDS), comprising administering to a subject in need thereof a therapeutically effective amount of an aldose reductase inhibitor (ARI). This disclosure relates to an aldose reductase inhibitor (ARI) for use in treating acute lung inflammation (ALI), and a method for treating acute lung inflammation (ALI), comprising administering to a subject in need thereof a therapeutically effective amount of an aldose reductase inhibitor (ART). This disclosure relates to an aldose reductase inhibitor (ARI) for use in treating acute lung injury, and a method for treating acute lung injury, comprising administering to a subject in need thereof a therapeutically effective amount of an aldose reductase inhibitor (ARI). In any such use or method, the subject to be treated can have an infection and the ARDS, ALI or acute lung injury is related to or caused by the infection.

This disclosure also relates to an aldose reductase inhibitor (ARI) for use in treating cardiac injury related to or caused by an infection, and a method for treating cardiac injury related to or caused by an infection, comprising administering to a subject in need thereof a therapeutically effective amount of an aldose reductase inhibitor (ARI).

This disclosure also relates to an aldose reductase inhibitor (ARI) for use in treating an infection, in decreasing pathogen burden in a subject with an infection, in inhibiting pathogen replication in a subject with an infection. This disclosure also relates to methods for treating an infection, decreasing pathogen burden in a subject with an infection, or inhibiting pathogen replication in a subject with an infection, comprising administering to a subject with an infection a therapeutically effective amount of an aldose reductase inhibitor (ARI).

In any of the uses or methods disclosed herein, the subject to be can have an infection. The infection can be a viral infection. In embodiments of the uses and methods described herein, the viral infection is infection with a herpes virus, such as is herpes simplex virus or cytomegalovirus. In embodiments of the uses and methods described herein, the viral infection is infection with a respiratory virus. The respiratory virus can be a respiratory syncytial virus (RSV), rhinovirus, parainfluenza virus (PIV), adenovirus, metapneumovirus, enterovirus, bocavirus, influenza virus or coronavirus. In embodiments of the uses and methods described herein, the infection is an influenza virus. In embodiments of the uses and methods described herein, the the infection is a coronavirus, such as SARS-CoV-1, SARS-CoV-2 or MERS-CoV.

In embodiments of the uses and methods disclosed herein, the infection is a bacterial infection. The bacterial infection can be infection with one or more of Streptococcus pneumoniae, Group B streptococci, Group A strptococci, Staphylococcus aureus, Hemophilus influenzae, Nisseria sp., Enterococcus sp., Mycoplasma pneumoniae, Chlamydia pneumoniae, Mycobacterium tuberculosis, and Mycobacterium avium.

In embodiments of the uses and methods described herein, the infection is a fungal infection. The fungal infection can be infection with one or more of Aspergillus sp., Blastomyces sp., Cryptococcus sp. In embodiments of the uses and methods described herein, the infection is a parasitic infection. The parasitic infection can be an infection with one or more of Pneumocystis jiroveci, Plasmodium falciparum or Plasmodium vivax. The subject to be treated according to any of the uses or methods disclosed herein can has sepsis.

In the disclosed uses and methods or treating cardiac injury related to or caused by an infection, the cardiac injury can be infection related or infection caused cardiomyopathy, infection related or infection caused myocarditis or infection related or infection causes acute cardiac injury. In embodiments, subject to be treated has elevated troponin (e.g., high-sensitivity cardiac troponin levels), NTproBNP and/or creatine kinase (e.g., CK-MB) levels. In embodiments, the the acute cardiac injury is characterized by elevated troponin levels (e.g., high-sensitivity cardiac troponin levels).

In the uses and method of this disclosure, the ARI can be zopolrestat or salt thereof, or epalrestat or salt thereof or a compound of any one of Formulas I-III or salt thereof. In some preferred embodiments, the ARI is a compound of Formula II or salt thereof. In particularly preferred embodiments, the ARI is

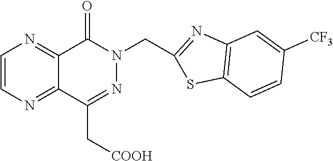 [SMILES: O=C(O)Cc1nn(Cc2nc3cc(C(F)(F)F)ccc3s2)c(=O)c2nccnc12]

or a salt thereof, which can, in embodiments, be administered in an amount of 1,500 mg orally twice a day.

In a particular aspect, this disclosure relates to an aldose reductase inhibitor (ARI) for use in treating acute respiratory distress syndrome (ARDS), acute lung inflammation (ALI) and/or acute lung injury, and a method for treating acute respiratory distress syndrome (ARDS), acure lung inflammation (ALI) and/or acute lung injury, comprising administering to a subject in need thereof a therapeutically effective amount of an aldose reductase inhibitor (ARI). The subject to be treated according to this aspect of the disclosure can have an infection and the ARDS, ALI or acute lung injury is related to or caused by the infection. The infection can be a viral infection. The viral infection can be infection with a herpes virus, such as is herpes simplex virus or cytomegalovirus. The viral infection can be infection with a respiratory virus. The respiratory virus can be a respiratory syncytial virus (RSV), rhinovirus, parainfluenza virus (Ply), adenovirus, metapneumovirus, enterovirus, bocavirus, influenza virus or coronavirus. In embodiments of the uses and methods of this aspect, the infection is an influenza virus. In embodiments of the uses and methods of this aspect, the infection is a coronavirus, such as SARS-CoV-1, SARS-CoV-2 or MERS-CoV. In this aspect of the uses and methods of the disclosure, the infection can be bacterial infection, a fungal infection or a parasitic infection, for example an infection with one or more bacteria, fungi and/or parasites as disclosed herein. The subject to be treated according to the uses or methods of this aspect can have sepsis. In the use and method of this aspect of the disclosure, the ARI can be zopolrestat or salt thereof, or epalrestat or salt thereof; or a compound of any one of Formulas I-III or salt thereof. In some preferred embodiments, the ARI is a compound of Formula II or salt thereof. In particularly preferred embodiments, the ARI is

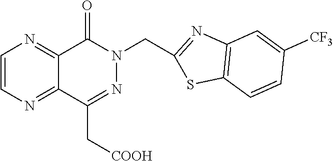 [SMILES: O=C(O)Cc1nn(Cc2nc3cc(C(F)(F)F)ccc3s2)c(=O)c2nccnc12]

or a salt thereof, which can, in embodiments, be administered in an amount of 1,500 mg orally twice a day.

In other particular aspects, this disclosure relates to use of an ARI in treating acute respiratory distress syndrome (ARDS), acute lung inflammation (ALI) or acute lung injury in a subject with influenza virus or a coronavirus disease, and a method for treating ARDS, ALE or acute lung injury related to or caused by influenza virus or a coronavirus disease, comprising administering to a subject with influenza or a coronavirus disease a therapeutically effective amount of an aldose reductase inhibitor (ARI). In other particular aspects, this disclosure relates to use of an ARI in treating cardiac injury related to or caused by influenza virus or a coronavirus disease, and a method for treating cardiac injury related to or caused by influenza virus or a coronavirus disease, comprising administering to a subject with influenza or a coronavirus disease a therapeutically effective amount of an aldose reductase inhibitor (ARI). In embodiments, the subject has influenza. In other embodiments, the subject has Severe Acute Respiratory Syndrome, Middle East Respiratory Syndrome or COVID-19. In particular embodiments, the subject has COVID-19. In the use and method of this aspect of the disclosure, the ARI can be zopolrestat or salt thereof, or epalrestat or salt thereof; or a compound of any one of Formulas I-III or salt thereof. In some preferred embodiments, the ARI is a compound of Formula II or salt thereof. In particularly preferred embodiments, the ARI is

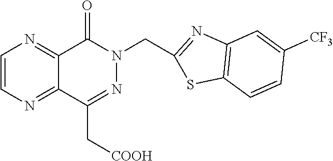 [SMILES: O=C(O)Cc1nn(Cc2nc3cc(C(F)(F)F)ccc3s2)c(=O)c2nccnc12]

or a salt thereof, which can, in embodiments, be administered in an amount of 1,500 mg orally twice a day.

This disclosure also relates to use of an aldose reductase inhibitor for treatment of a disease or condition described herein. This disclosure also relates to an aldose reductase inhibitor for use in the manufacture of a medicament for treatment of a disease or condition described herein. This disclosure also relates to a pharmaceutical composition for treatment of a disease or condition described herein that comprises an aldose reductase inhibitor as an active agent.

Typically, the inhibitor of aldose reductase is administered at least once a day in the practice of the methods disclosed herein.

### DETAILED DESCRIPTION

This disclosure relates to the use of an aldose reductase inhibitor (ARI) for the treatment of acute respiratory distress syndrome (ARDS), acute lung inflammation (ALI) and/or acute lung injury, including ARDS, ALI and acute lung injury in a subject that has an infection and wherein the ARDS, ALI and/or acute lung injury is related to are caused by the infection. This disclosure also relates to the use of aldose reductase inhibitor (ARI) for the treatment of cardiac injury (e.g., acute cardiac injury), such as cardiac injury that is related to our caused by an infection. The inventor has discovered that ARIs can be used to successfully treat cardiopulmonary sequela of infection, including in patients with critical influenza or coronavirus disease, such as SARS, MERS and COVID-19.

This disclosure also relates to the use of an aldose reductase inhibitor (ARI) for the treatment of infection, reducing pathogen burden and/or inhibiting pathogen replication, in a subject that has an infection.

The inventor has discovered that surprisingly ARIs have anti-viral activity and can be administered to treat infection, to reduce pathogen burden and/or to inhibit pathogen replication.

Without wishing to be bound by any particular theory or mechanism, it is believed that infectious pathogens, such as influenza virus and coronavirus (e.g., SARS-CoV-1, SARS-CoV-2, MERS-CoV) cause pathogen-related or pathogen-caused oxidative stress. Such oxidative stress is believed to be a result, at least in part, of the activity of AR, and includes the downstream production of reactive oxygen species (ROS) and advanced glycation end products (AGEs), which can lead to the activation of inflammatory pathways including the JNK/IL-6 pathway and modulation of other inflammatory pathways including the JAK/STAT, PI3K/AKT, mTOR, phospholipase C and protein kinase C pathways, and mediate oxidative damage in cardiomyocytes and oxidative-induced cardiomyopathies. These inflammatory pathways can also cause changes in the infected subject (e.g., in the lungs) that are conducive to pathogen replication (viral replication) and increased pathogen burden (viral burden). Inhibition of AR and resulting oxidative stress and ROS, can reduce activation of inflammatory pathways including the JNK/IL-6 pathway and other inflammatory pathways. It is believed that this can then reduce the downstream increase in alveolar-capillary permeability and edema formation that underlie the pathogenesis of ARDS, ALI and acute lung injury. It is believed that inhibiting aldose reductase using an ARI as described herein can effectively provide anti-viral therapy by inhibiting AR-mediated inflammatory changes.

Where a range of values is provided in this disclosure, it is intended that each intervening value between the upper and lower limit of that range and any other stated or intervening value in that stated range is encompassed within the disclosure. For example, if a range of 1 μM to 8 μM is stated, it is intended that 2 μM, 3 μM, 4 μM, 5 μM, 6 μM, and 7 μM are also explicitly disclosed, as well as the range of values greater than or equal to 1 μM and the range of values less than or equal to 8 μM.

The singular forms “a,” “an,” and “the” include plural referents unless the context clearly dictates otherwise. Thus, for example, reference to a “compound of Formula I” includes a single compound as well as two or more of the same or different compounds; reference to an “excipient” includes a single excipient as well as two or more of the same or different excipients, and the like.

The word “about” means a range of plus or minus 10% of that value, e.g., “about 50” means 45 to 55, “about 25,000” means 22,500 to 27,500, etc., unless the context of the disclosure indicates otherwise, or is inconsistent with such an interpretation. For example in a list of numerical values such as “about 49, about 50, about 55, “about 50” means a range extending to less than half the interval(s) between the preceding and subsequent values, e.g., more than 49.5 to less than 52.5. Furthermore, the phrases “less than about” a value or “greater than about” a value should be understood in view of the definition of the term “about” provided herein.

A “subject” can be any animal, particularly a mammal, and including, but not limited to, humans, domestic animals, such as feline or canine subjects, farm animals, such as but not limited to bovine, equine, caprine, ovine, avian and porcine subjects, wild animals (whether in the wild or in a zoological garden), research or laboratory animals, such as mice, rats, rabbits, goats, sheep, pigs, dogs, cats, etc., avian species, such as chickens, turkeys, songbirds, and the like. Preferably, the “subject” is a human.

As used herein, the terms “treatment” and “treating” in reference to medical therapy refer to therapy to reduce, resolve, stop progression of, or stabilize infection, such as respiratory infection, ARDS, ALI, acute lung injury and/or cardiac injury. Accordingly, treatment can be therapy for reducing, arresting or delaying the signs, symptoms, characteristics and/or underlying pathogenic pathways of ARDS, ALI, acute lung injury and/or cardiac injury, such as elevated levels of cytokines, increased alveolar-capillary permeability, pulmonary edema, myocardial injury, cardiomyopathy, arrhythmia and myocarditis. In embodiments, treatment can stop an increase in or reduce pathogen burden in an infected subject and/or inhibit pathogen replication.

As used herein “a therapeutically effective amount” is an amount of a compound that is sufficient to achieve the desired therapeutic effect under the conditions of administration, such as an amount that reduces AR activity, reduces ROS and/or AGEs, reduces or stabilizes cytokine levels, alveolar-capillary permeability, pulmonary edema, reduces myocardial injury, cardiomyopathy, arrhythmia and/or myocarditis. In embodiments, the effective amount reduces pathogen burden, inhibits pathogen replication and/or inhibits pathogen infectivity. In some examples, the therapeutically effective amount is an amount sufficient to reduce intracellular aldose reductase activity at least by about 5%, at least about 10%, at least about 20%, at least about 30%, at least about 40%, at least about 50%, at least about 60%, at least about 70%, at least about 80%, at least about 90%, at least about 95%, at least about 99%, or more, e.g., about 100% (e.g., compared to pre-treatment level). The actual amount administered can be determined by an ordinarily skilled clinician based upon, for example, the subjects age, weight, sex, general heath and tolerance to drugs, severity of disease, dosage form selected, route of administration and other factors. The amount of an AR inhibitor that is administered systemically can be from about 0.5 to about 60 mg/kg body weight per day, for example, a human can be administered a 1500 mg dose of ARI orally twice a day.

In order to provide a complete, concise and clear description of the various embodiments, this disclosure includes descriptions of various components, groups of components, ranges and other elements of the broader disclosure. It is intended that such elements can be variously combined to provide additional embodiments of the disclosure. It is also intended that any disclosed features (e.g., substituent, analog, compound, structure, component) including individual members of any disclosed group, including any sub-ranges or combinations of sub-ranges within the group, may be excluded from the disclosure or any embodiments of the disclosure for any reason.

### I. Methods and Uses of ARIs

This disclosure relates to methods for the treatment of acute respiratory distress syndrome (ARDS), acute lung inflammation (ALI) and/or acute lung injury, comprising administering to a subject in need thereof a therapeutically effective amount of an ARI. ARDS, ALI and/or acute lung injury are major causes of morbidity and mortality in critically ill patients, and can be caused by infection (e.g, viral, bacterial or fungal pneumonia), inflammatory diseases, trauma, or inhalation of toxic or irritating substances, such as chemicals and smoke. The methods disclosed herein include treatment of ARDS, ALI and acute lung injury in a subject that has an infection and wherein the ARDS, ALI and/or acute lung injury is related to or is caused by the infection. This disclosure also relates to the use of aldose reductase inhibitor (ARI) for the treatment of cardiac injury (e.g., acute cardiac injury), comprising administering to a subject in need thereof a therapeutically effective amount of an ARI.

ARDS, ALI, acute lung injury and/or cardiac injury that is “related to” e.g. a pathogen, may not be directly caused by that pathogen but be a sequelae of infection with the pathogen. For example, the increased alveolar-capillary permeability and edema formation that underlie the pathogenesis of ARDS, ALI and acute lung injury may not be caused directly by infection (e.g., with influenza virus or coronavirus, such as SARS-CoV-1, SARS-CoV-2, MERS-CoV) of alveolar capillary cells (e.g., endothelial cells) but result from the subjects inflammatory response to infection. In another example, ARDS, ALI and/or acute lung injury may be related to an inflammatory condition in another part of the body, such as pancreatitis.

In particular aspects, the methods for treating ARDS, ALI and/or acute lung injury are practiced on a subject who has an infection. The ARDS, ALI and/or acute lung injury can be related to or caused by the infection. Similarly, in certain aspects the methods for treating cardiac injury are practiced on a subject who has an infection. The cardiac injury can be related to or caused by the infection.

The infection can be a viral infection, a bacterial infection, a fungal infection or a parasitic infection. Typically, the infection causes oxidative stress and/or an immune response, which results in cardiopulmonary tissue damage. In some aspects, the subject to be treated has pneumonia, including viral pneumonia, bacterial pneumonia, fungal pneumonia or parasitic pneumonia.

The subject to be treated may have a viral infection. For example, the subject can be infected with a respiratory virus or a herpes virus. Exemplary respiratory viruses include respiratory syncytial virus (RSV), rhinovirus, parainfluenza virus (PIV), adenovirus, metapneumovirus, enterovirus, bocavirus, influenza virus and coronavirus. In particular practices, the subject is infected with an influenza virus (e.g., influenza type A, influenza type B). The subject may have signs and/or symptoms of influenza. In other practices, the subject is infected with a coronavirus (e.g., SARS-CoV-1, SARS-CoV-2, MERS-CoV). The subject may have signs and/or symptoms of coronavirus disease (e.g., SARS, MERS, COVID-19). The subject may be infected with a herpesvirus, such as herpes simplex virus (e.g., HSV1, HSV2) or cytomegalovirus.

The subject to be treated may have a bacterial infection. Exemplary bacterial infections include infections with Streptococcus pneumoniae, Group B streptococci, Group A streptococci, Staphylococcus aureus, Hemophilus influenzae, Nisseria sp., Enterococcus sp., Mycoplasma pneumoniae, Chlamydia pneumoniae, Mycobacterium tuberculosis and Mycobacterium avium.

The subject to be treated may have a fungal infection. Exemplary fungal infections include infections with Aspergillus sp., Blastomyces sp., Cryptococcus sp.

The subject to be treated may have a parasitic infection. Exemplary parasitic infections include infections with Pneumocystis jiroveci, Plasmodium falciparum or Plasmodium vivax.

In some practices of the methods for treating ARDS, ALI, acute lung injury and/or cardiac injury (e.g., acute cardiac injury), the subject to be treated has sepsis.

In some practices of the methods for treating ARDS, ALI, acute lung injury and/or cardiac injury (e.g., acute cardiac injury), the subject to be treated has SARS.

In some practices of the methods for treating ARDS, ALI, acute lung injury and/or cardiac injury (e.g., acute cardiac injury), the subject to be treated has MERS.

In some practices of the methods for treating ARDS, ALI, acute lung injury and/or cardiac injury (e.g., acute cardiac injury), the subject to be treated has COVID-19.

In some practices of the methods for treating ARDS, ALI, acute lung injury and/or cardiac injury (e.g., acute cardiac injury), the subject to be treated has influenza.

In some practices of the methods for treating ARDS, ALI, acute lung injury and/or cardiac injury (e.g., acute cardiac injury), the subject to be treated has underlying cardiovascular disease, such as cardiomyopathy.

In some practices of the method of treating cardiac injury (e.g., acute cardiac injury), the subject has cardiac injury that is caused by or related to infection or an inflammatory disease. For example, the cardiac injury can be infection related or infection caused myocarditis, cardiomyopathy and/or acute cardiac injury (e.g., acute myocardial damage). Acute cardiac injury can be determined using a variety of methods that are routine and well-known in the art, such as levels of biomarkers of acute cardiac injury. (See, e.g., Bodor GS. Biochemical Markers of Myocardial Damage. EJIFCC. 2016;27(2):95-111. Published 2016 Apr. 20) For example, acute cardiac injury can be determined by elevated levels of troponin (e.g., high-sensitivity cardiac troponin), NTproBNP and/or creatine kinase (e.g., CK-MB).

This disclosure relates to the use of an aldose reductase inhibitor (ARI) for the treatment of infection, reducing pathogen burden (e.g., viral burden) and/or inhibiting pathogen replication (e.g., viral replication), in a subject in need thereof (e.g., that has an infection), comprising administering to a subject in need thereof a therapeutically effective

In particular aspects, the methods for treating infection, reducing pathogen burden and/or inhibiting pathogen replication are practiced on a subject who has an infection, such as a viral infection. The infection can be a viral infection, a bacterial infection, a fungal infection or a parasitic infection. Typically, the infection causes oxidative stress and/or an immune response, which can be conducive to pathogen replication and/or increased pathogen burden, such as influenza or coronavirus (e.g. SARS-CoV-1, SARS-CoV2, MERS) replication and/or burden.

In some aspects, the subject to be treated has pneumonia, including viral pneumonia, bacterial pneumonia, fungal pneumonia or parasitic pneumonia.

The subject to be treated can have a viral infection. For example, the subject can be infected with a virus as disclosed herein, such as respiratory virus or a herpes virus. In particular practices, the subject is infected with an influenza virus (e.g., influenza type A, influenza type B). The subject may have signs and/or symptoms of influenza. In other practices, the subject is infected with a coronavirus (e.g., SARS-CoV-1, SARS-CoV-2, MERS-CoV). The subject may have signs and/or symptoms of coronavirus disease (e.g., SARS, MERS, COVID-19). The subject may be infected with a herpesvirus, such as herpes simplex virus (e.g., HSV1, HSV2) or cytomegalovirus.

The subject to be treated can have a bacterial infection, such as an infection by any of the bacteria disclosed herein. The subject to be treated can have a fungal infection, such as an infection by any of the fungi disclosed herein. The subject to be treated can have a parasitic infection, such as an infection by any of the parasites disclosed herein.

In some practices of the methods for treating infection, reducing pathogen burden and/or inhibiting pathogen replication, the subject to be treated has sepsis.

In some practices of the methods for treating infection, reducing pathogen burden and/or inhibiting pathogen replication, the subject to be treated has SARS. In some practices of the methods for treating infection, reducing pathogen burden and/or inhibiting pathogen replication, the subject to be treated has MERS. In some practices of the methods for treating infection, reducing pathogen burden and/or inhibiting pathogen replication, the subject to be treated has COVID-19. In some practices of the methods for treating infection, reducing pathogen burden and/or inhibiting pathogen replication, the subject to be treated has influenza. In some practices of the methods for treating infection, reducing pathogen burden and/or inhibiting pathogen replication, the subject to be treated has underlying cardiovascular disease, such as cardiomyopathy. In some practices of the method of treating infection, reducing pathogen burden and/or inhibiting pathogen replication, the subject has cardiac injury that is caused by or related to infection.

The AR inhibitor administered in accordance with the disclosed methods can be any compound that inhibits AR activity, such as a small molecule compound (e.g., having a size of 5 kDa or less), a biologic agent (e.g., an inhibitory RNA directed against aldose reductase) or a combination thereof.

In one example, the disclosed methods comprise administering to a subject in need thereof a therapeutically effective amount of zopolrestat.

In another example, the disclosed methods comprise administering to a subject in need thereof a therapeutically effective amount of epalrestat.

In other examples, the disclosed methods comprises administering to a subject in need thereof a therapeutically effective amount of an aldose reductase inhibitor, that is not ponalrestat, epalrestat, sorbinil or sorbinol, imirestat, AND-138, CT-112, zopolrestat, zenarestat, BAL-AR18, AD-5467, M-79175, tolrestat, alconil, statil, berberine or SPR-210.

In some preferred aspects, the disclosed methods comprise administering to a subject in need thereof a therapeutically effective amount of a compound of any one of Formulas I-III. In other preferred aspects, the disclosed methods comprise administering to a subject in need thereof a therapeutically effective amount of a compound of Formulas II or Formula III. A preferred compound for use in the methods disclosed herein is Compound A.

The methods disclosed herein can be practiced by administering a single dosage or single administration (e.g., as a single injection or deposition) of one or more AR inhibitors, but typically involve an administration regimen under which an AR inhibitor is administered at least once daily for the desired course of treatment. For example, the AR inhibitor can be administered once daily, twice daily, three times daily, four times daily or more to a subject in need thereof for a period of from about 2 to about 28 days, or from about 7 to about 10 days, or from about 7 to about 15 days, or longer. The disclosed methods include long-term administration regimens, for example with the administration of an AR inhibitor at least once daily for a period of weeks, months, years or decades.

### II. AR Inhibitors

Suitable small molecule AR inhibitors are known in the art and are disclosed herein. Small molecule AR inhibitors include ponalrestat, sorbinil, sorbinol, imirestat, AND-138, CT-112, zenarestat, BAL-AR18, AD-5467, M-79175, tolrestat, alconil, statil, berberine, SPR-210, zopolrestat, epalrestat, the compounds disclosed in U.S. Pat. Nos. 8,916,563, 9,650,383, 10,150,779 and the compounds disclosed herein. Preferred AR inhibitors for use in the invention include zopolrestat, epalrestat, the compounds disclosed in U.S. Pat. Nos. 8,916,563, 9,650,383, 10,150,779 and the compounds disclosed herein. The AR inhibitors can be administered in any suitable molecular form including pharmaceutically acceptable salts, solvates, prodrugs, and compounds that contain stable isotopic forms of one or more atoms, e.g., deuterium in place of hydrogen.

### AR Inhibitors of Formulas I and II

In one example, the AR inhibitor is a compound of Formula (I) or pharmaceutically acceptable salts, prodrugs and solvates thereof,

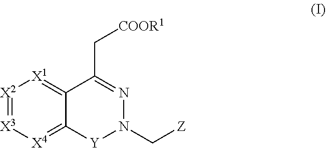

wherein,

R¹ is H, (C₁-C₆)-alkyl, (C₁-C₆)-hydroxyalkyl, or (C₁-C₆)-aminoalkyl;

X¹ is N or CR³;

X² is N or CR⁴;

X³ is N or CR⁵;

X⁴ is N or CR⁶; with the proviso that two or three of X¹, X², X³, or X⁴ are N;

Y is a bond, C═O, C═S, C═NH, or C═N(C₁-C₄)-alkyl;

Z is

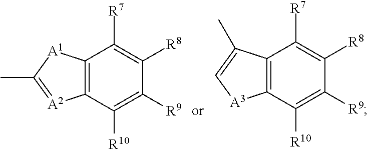

A¹ is NR¹¹, O, S or CH₂;

A² is N or CH;

A³ is NR¹¹, O, or S;

R³ through R¹⁰ are independently hydrogen, halogen, cyano, acyl, haloalkyl, haloalkoxy, haloalkylthio, trifluoroacetyl, (C₁-C₄)-alkyl, (C₁-C₄)-alkoxy, (C₁-C₄)-alkylthio, (C₁-C₄)-alkylsulfinyl, or (C₁-C₄)-alkylsulfonyl; or two of R³through R⁶ or two of R⁷ through R¹⁰ taken together are (C₁-C₄)-alkylenedioxy; and

R¹¹ is hydrogen, C₁-C₄ alkyl, or C(O)O—(C₁-C₄)-alkyl.

It will be recognized by those of skill in the art that the designation of Z is

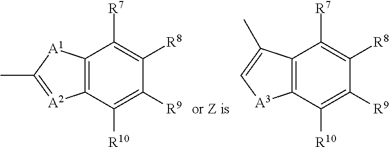

indicates that when Z is

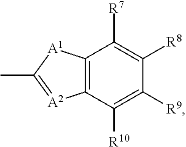

the compounds of formula (I) encompass

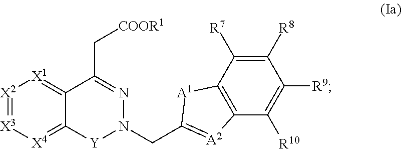

and when Z is

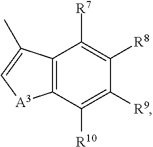

the compounds of formula (I) encompass

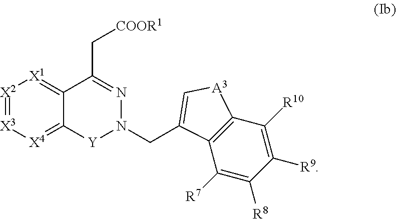

In certain embodiments, R¹ is hydrogen or (C₁-C₆)-alkyl. In certain embodiments, R¹ is hydrogen. In certain embodiments, R¹ is (C₁-C₆)-alkyl. In certain embodiments, R¹ is tert-butyl.

In certain embodiments, R³ through R¹⁰ are independently hydrogen, halogen or haloalkyl. In certain embodiments, R³ through R¹⁰ are independently hydrogen, halogen or trihaloalkyl.

In certain embodiments, R³ through R⁶ are hydrogen.

In certain embodiments, R⁷ through R¹⁰ are independently hydrogen, halogen or haloalkyl. In certain embodiments, R⁷ through R¹⁰ are independently hydrogen, halogen or trihaloalkyl.

In certain embodiments, R⁷ and R¹⁰ are hydrogen.

In certain embodiments, R⁸ is hydrogen, halogen or haloalkyl. In certain embodiments, R⁸ is hydrogen. In certain embodiments, R⁸ is halogen. In certain embodiments, R⁸ is haloalkyl.

In certain embodiments, R⁹ is hydrogen, halogen or haloalkyl. In certain embodiments, R⁹ is hydrogen. In certain embodiments, R⁹ is halogen. In certain embodiments, R⁹ is haloalkyl.

In certain embodiments, Y is C═O, C═S, C═NH, or C═N(C₁-C₄)-alkyl. In certain embodiments, Y is or C═S. In certain embodiments, Y is C═O. In certain embodiments, Y is C═S. In certain embodiments, Y is C═NH, or C═N(C₁-C₄)-alkyl.

In certain embodiments, A¹ is NR¹¹, S or CH₂. In certain embodiments, A¹ is is NR¹¹ or O. In certain embodiments, A¹ is NR¹¹ or S. In certain embodiments, A¹ is NR¹¹. In certain embodiments, A¹ is O. In certain embodiments, A¹ is S.

In certain embodiments, A² is N or CH. In certain embodiments, A¹ is N. In certain embodiments, A¹ is CH.

In certain embodiments, A³ is O or S. In certain embodiments, A³ is O. In certain embodiments, A³ is S.

In certain embodiments, X¹ and X⁴ are nitrogen.

In certain embodiments, X¹ and X² are nitrogen.

In certain embodiments, X¹ and X³ are nitrogen.

In certain embodiments, X² and X³ are nitrogen.

In certain embodiments, X² and X⁴ are nitrogen.

In certain embodiments, X³ and X⁴ are nitrogen.

In certain embodiments, Z is

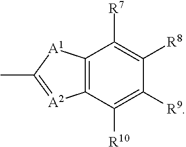

In certain embodiments, Z is

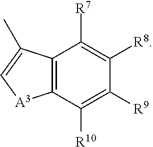

In certain embodiments, R¹ is hydrogen or (C₁-C₆)-alkyl;

X¹ and X⁴ are N;

X² is CR⁴;

X³ is CR⁵;

Y is C═O;

Z is

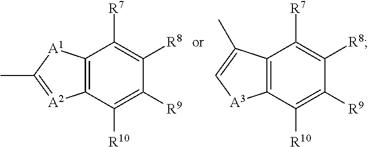

A¹ is NR¹¹, O, or S;

A² is N;

A³ is O, or S;

R⁴ and R⁵ are hydrogen;

R⁷ through R¹⁰ are independently hydrogen, halogen, cyano, acyl, haloalkyl, haloalkoxy, haloalkylthio, (C₁-C₄)-alkyl, (C₁-C₄)-alkoxy, (C₁-C₄)-alkylthio, (C₁-C₄)-alkylsulfinyl, or (C₁-C₄)-alkyl sulfonyl; and

R¹¹ is hydrogen, C₁-C₄ alkyl, or C(O)O—(C₁-C₄)-alkyl.

In certain embodiments, R¹ is hydrogen or tert-butyl;

X¹ and X⁴ are N;

X² is CR⁴;

X³ is CR⁵;

Y is C═O;

Z is

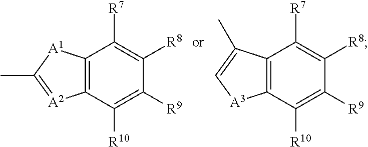

A¹ is NR¹¹, O or S;

A² is N;

A³ is O or S;

R⁴ and R⁵ are hydrogen;

R⁷ through R¹⁰ are independently hydrogen, halogen, or haloalkyl; and

R¹¹ is hydrogen, (C₁-C₄)-alkyl, or C(O)O-tert-butyl.

In certain embodiments, R¹ is hydrogen or tert-butyl;

X¹ and X⁴ are N;

X² is CH;

X³ is CH;

Y is C═O;

Z is

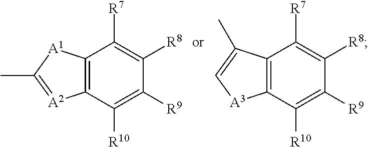

A¹ is NR¹¹, O or S;

A² is N;

A³ is O or S;

R⁷, R⁸ and R¹⁰ are independently hydrogen, halogen, or haloalkyl;

R⁹ is halogen, or haloalkyl; and

R¹¹ is hydrogen or methyl.

In certain embodiments, R¹ is hydrogen or tert-butyl;

X¹ and X⁴ are N;

X² is CH;

X³ is CH;

Y is C═O;

Z is

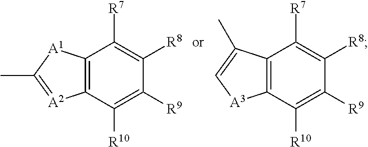

A¹ is NR¹¹, O or S;

A² is N;

A³ is O or S;

R⁷, R⁸ and R¹⁰ are independently hydrogen, halogen, or haloalkyl;

R⁹ is chlorine, or trifluoromethyl; and

R¹¹ is hydrogen or methyl.

In certain embodiments, the AR inhibitor is a compound of Formula (II) or pharmaceutically acceptable salt or solvate thereof:

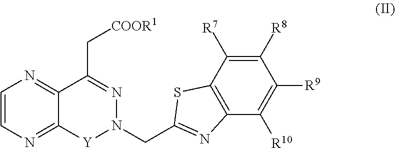

Wherein R¹, R⁷-R⁹ and Y are as described in Formula I, and preferable wherein R¹ is hydrogen or (C₁-C₆)-alkyl and Y is C═O. Exemplary compounds of Formula II include the following and salts thereof:

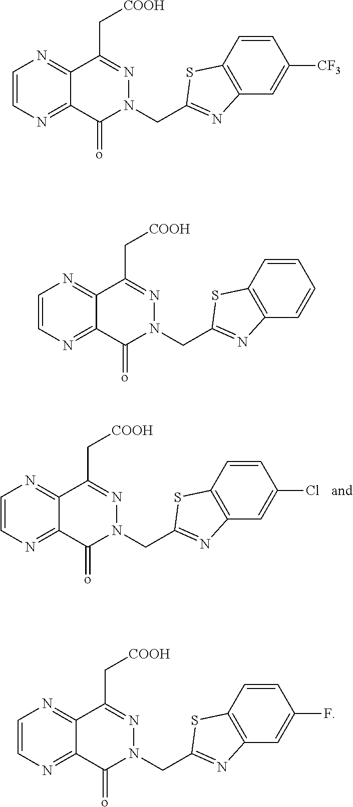

Compounds of Formula III

The AR inhibitors can be a compound of Formula (III) or pharmaceutically acceptable salts, pro-drugs and solvates thereof,

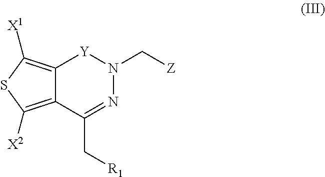

wherein,

R¹ is CO₂R² or CO₂⁻X⁺;

R² is H, (C₁-C₆)-alkyl, (C₁-C₆)-hydroxyalkyl, or (C₁-C₆)-aminoalkyl;

X¹ is H or halogen;

X² is H or halogen;

Y is a bond, C═O, C═S, C═NH, or C═N(C₁-C₄)-alkyl;

Z is

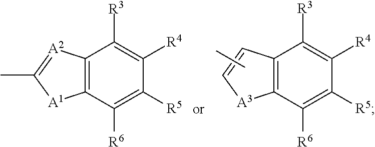

A¹ is NR⁷, O, S or CH₂;

A² is N or CH;

A³ is NR⁷, O, or S;

R³ through R⁶ are independently hydrogen, halogen, cyano, acyl, haloalkyl, haloalkoxy, haloalkylthio, trifluoroacetyl, (C₁-C₄)-alkyl, (C₁-C₄)-alkoxy, (C₁-C₄)-alkylthio, (C₁-C₄)-alkylsulfinyl, or (C₁-C₄)-alkylsulfonyl;

R⁷ is hydrogen, C₁-C₄ alkyl, or C(O)O—(C₁-C₄)-alkyl; and

X⁺ is a counter ion.

It will be recognized by those of skill in the art that the designation of

Z is

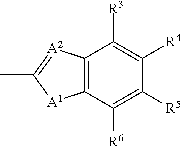

### or Z is

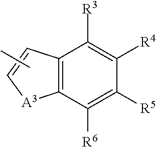

indicates that when Z is

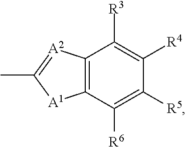

the compounds of Formula (III) are understood to encompass

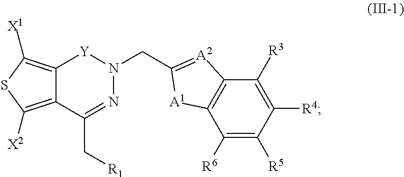

and when Z is

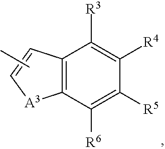

the compounds of Formula (I) are understood to encompass

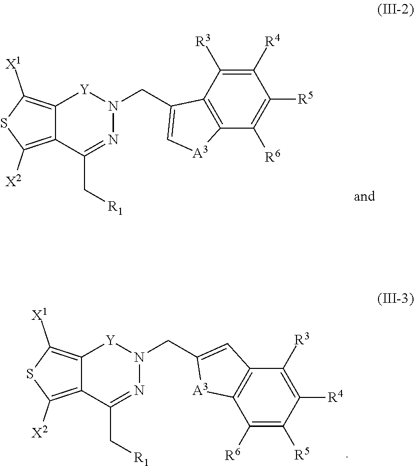

In certain embodiments, R¹ is CO₂R² or CO₂⁻X⁺. In certain embodiments, R¹ is CO₂R². In certain embodiments, R¹ is CO₂⁻X⁺.

In certain embodiments, R² is hydrogen or (C₁-C₆)-alkyl. In certain embodiments, R² is hydrogen or (C₁-C₄)-alkyl. In certain embodiments, R² is hydrogen or (C₁-C₃)-alkyl. In certain embodiments, R² is hydrogen, methyl, or ethyl. In certain embodiments, R² is hydrogen or methyl. In certain embodiments, R² is methyl or ethyl. In certain embodiments, R² is methyl. In certain embodiments, R² is hydrogen. In certain embodiments, R² is (C₁-C₆)-alkyl. In certain embodiments, R² is (C₁-C₆)-n-alkyl. In certain embodiments, R² is (C₁-C₂)-alkyl. In certain embodiments, R² is (C₁-C₃)-alkyl. In certain embodiments, R² is (C₁-C₄)-alkyl. In certain embodiments, R² is tert-butyl.

In certain embodiments, R³ through R⁶ are independently hydrogen, halogen, cyano, acyl, haloalkyl, haloalkoxy, haloalkylthio, trifluoroacetyl, (C₁-C₄)-alkyl, (C₁-C₄)-alkoxy, (C₁-C₄)-alkylthio, (C₁-C₄)-alkylsulfinyl, or (C₁-C₄)-alkylsulfonyl.

In certain embodiments, R³ through R⁶ are independently hydrogen, halogen or haloalkyl. In certain embodiments, R³ through R⁶ are independently hydrogen, halogen or trihaloalkyl.

In certain embodiments, R³ and R⁶ are hydrogen. In certain embodiments, R³, R⁵, and R⁶ are hydrogen.

In certain embodiments, R⁴ is hydrogen, halogen or haloalkyl. In certain embodiments, R⁴ is hydrogen. In certain embodiments, R⁴ is halogen. In certain embodiments, R⁴ is haloalkyl. I n certain embodiments, R⁴ is CF₃.

In certain embodiments, R³ through R⁶ are hydrogen. In certain embodiments, R³, R⁵, R⁶ are hydrogen and R⁴ is halogen or haloalkyl. In certain embodiments, R³, R⁵, R⁶ are hydrogen and R⁴ is haloalkyl. In certain embodiments, R³, R⁵, R⁶ are hydrogen and R⁴ is CF₃. In certain embodiments, R³, R⁵, R⁶ are hydrogen and R⁴ is halogen. In certain embodiments, R³, R⁵, R⁶ are hydrogen and R⁴ is F. In certain embodiments, R³, R⁵, R⁶ are hydrogen and R⁴ is Cl.

In certain embodiments, Y is C═O, C═S, C═NH, or C═N(C₁-C₄)-alkyl. In certain embodiments, Y is C═O or C═S. In certain embodiments, Y is C═O. In certain embodiments, Y is C═S. In certain embodiments, Y is C═NH, or C═N(C₁-C₄)-alkyl.

In certain embodiments, A¹ is NR⁷, O, S or CH₂. In certain embodiments, A¹ is NR⁷, O, or S. In certain embodiments, A^{1l} is NR⁷, S or CH₂. In certain embodiments, A¹ is NR⁷ or O. In certain embodiments, A¹ is NR' or S. In certain embodiments, A¹ is NR⁷. In certain embodiments, A¹ is O. In certain embodiments, A¹ is S.

In certain embodiments, A² is N or CH. In certain embodiments, A² is N. In certain embodiments, A² is CH.

In certain embodiments, A³ is NR⁷, O, or S. In certain embodiments, A³ is O. In certain embodiments, A³ is S. In certain embodiments, A³ is NR⁷.

In certain embodiments, X¹ and X² are hydrogen.

In certain embodiments, X¹ and X² are halogen. In certain embodiments, X¹ and X² are Cl.

In certain embodiments, X¹ and X² are independently hydrogen or halogen. In certain embodiments, X¹ is hydrogen and X² is Cl. In certain embodiments, X¹ is Cl and X² is hydrogen.

In certain embodiments, Z is

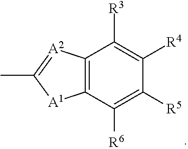

In certain embodiments, Z is

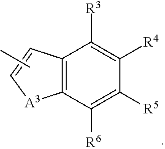

In certain embodiments, R⁷ is hydrogen, C₁-C₄ alkyl, or C(O)O—(C₁-C₄)-alkyl. In certain embodiments, R⁷ is hydrogen. In certain embodiments, R⁷ is C₁-C₄ alkyl. In certain embodiments, R⁷ is C₁-C₃ alkyl. In certain embodiments, R⁷ is C₁-C₂ alkyl. In certain embodiments, R⁷ is C₁-C₄ n-alkyl. In certain embodiments, R⁷ is C₁-C₃ n-alkyl. In certain embodiments, R⁷ is C(O)O—(C₁-C₄)-alkyl. In certain embodiments, R⁷ is C(O)O—(C₁-C₃)-alkyl. In certain embodiments, R⁷ is C(O)O—(C₁-C₂)-alkyl. In certain embodiments, R⁷ is C(O)O—(C₁-C₄)-n-alkyl. In certain embodiments, R⁷ is C(O)O-(C₁-C₃)-n-alkyl.

In certain embodiments, R¹ is CO₂R²;

R² is H or (C₁-C₆)-alkyl;

X¹ is H;

X² is H;

Y is C═O;

Z is

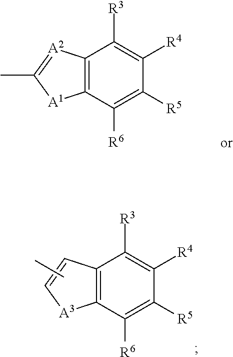

A¹ is NR⁷, O, or S;

A² is N;

A³ is O or S;

R³ through R⁶ are independently hydrogen, halogen, cyano, acyl, haloalkyl, haloalkoxy, haloalkylthio, trifluoroacetyl, (C₁-C₄)-alkyl, (C₁-C₄)-alkoxy, (C₁-C₄)-alkylthio, (C₁-C₄)-alkylsulfinyl, or (C₁-C₄)-alkylsulfonyl; and

R⁷ is hydrogen, C₁-C₄ alkyl, or C(O)O—(C₁-C₄)-alkyl.

In certain embodiments, R¹ is CO₂R²;

R² is H or tert-butyl;

X¹ is H;

X² is H;

Y is C═O;

Z is

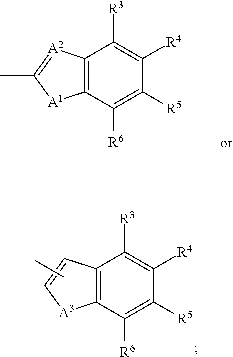

A¹ is NR⁷, O, or S;

A² is N;

A³ is O or S;

R⁶ through R⁶ are independently hydrogen, halogen, haloalkyl; and

R⁷ is hydrogen, C₁-C₄ alkyl, or C(O)O—(C₁-C₄)-alkyl.

In certain embodiments, R¹ is CO₂R²;

R² is H or tert-butyl;

X¹ is H;

X² is H;

Y is C═O;

Z is

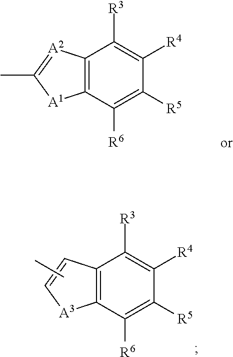

A¹ is NR⁷, O, or S;

A² is N;

A³ is O or S;

R³, R⁵, and R⁶ are hydrogen;

R⁴ is hydrogen, halogen, or haloalkyl; and

R⁷ is hydrogen, C₁-C₄ alkyl, or C(O)O—(C₁-C₄)-alkyl.

In certain embodiments, R¹ is CO₂R²;

R² is H or (C₁-C₆)-alkyl;

X¹ is halogen;

X² is halogen;

Y is C═O;

Z is

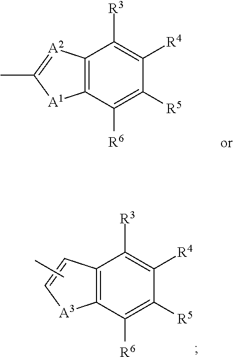

A¹ is NR⁷, O, or S;

A² is N;

A³ is O or S;

R³ through R⁶ are independently hydrogen, halogen, cyano, acyl, haloalkyl, haloalkoxy, haloalkylthio, trifluoroacetyl, (C₁-C₄)-alkyl, (C₁-C₄)-alkoxy, (C₁-C₄)-alkylthio, (C₁-C₄)-alkylsulfinyl, or (C₁-C₄)-alkylsulfonyl; and

R⁷ is hydrogen, C₁-C₄ alkyl, or C(O)O—(C₁-C₄)-alkyl.

In certain embodiments, R¹ is CO₂R²;

R² is H or tert-butyl;

X¹ is halogen;

X² is halogen;

Y is C═O;

Z is

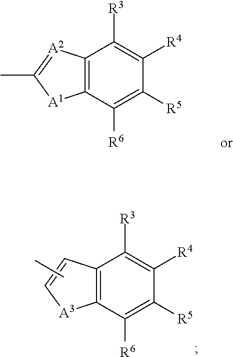

A¹ is NR⁷, O, or S;

A² is N;

A³ is O or S;

R³ through R⁶ are independently hydrogen, halogen, haloalkyl; and

R⁷ is hydrogen, C₁-C₄ alkyl, or C(O)O—(C₁-C₄)-alkyl.

In certain embodiments, R¹ is CO₂R²;

R² is H or tert-butyl;

X¹ is Cl;

X² is Cl;

Y is C═O;

Z is

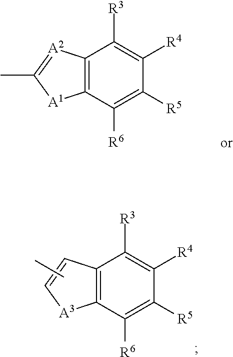

A¹ is NR⁷, O, or S;

A² is N;

A³ is O or S;

R³ through R⁶ are independently hydrogen, halogen, haloalkyl; and

R⁷ is hydrogen, C₁-C₄ alkyl, or C(O)O—(C₁-C₄)-alkyl.

In certain embodiments, le is CO₂R²;

R² is H or tert-butyl;

X¹ is Cl;

X² is Cl;

Y is C═O;

Z is

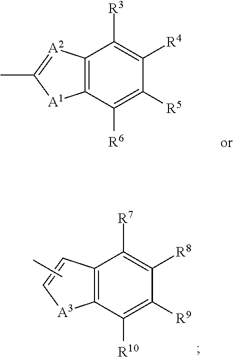

A¹ is NR⁷, O, or S;

A² is N;

A³ is O or S;

R³, R⁵, and R⁶ are hydrogen;

R⁴ is hydrogen, halogen, or haloalkyl; and

R⁷ is hydrogen, C₁-C₄ alkyl, or C(O)O—(C₁-C₄)-alkyl.

In certain embodiments, the compound of Formula (III) is selected from the group consisting of:

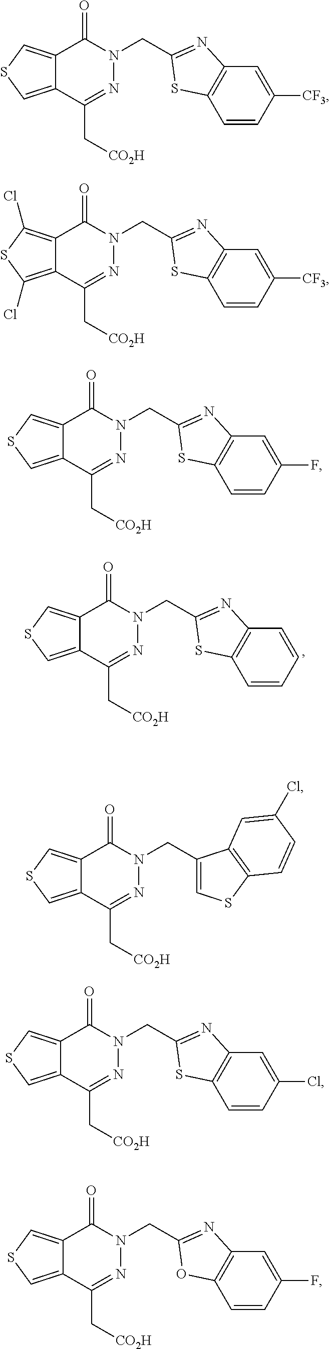

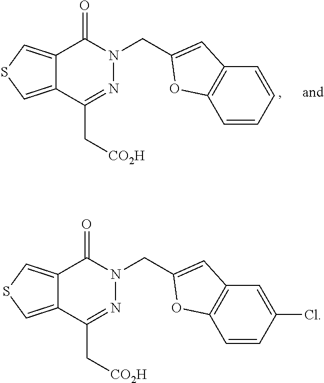 [SMILES: O=C(O)Cc1nn(Cc2cc3ccccc3o2)c(=O)c2cscc12 ; O=C(O)Cc1nn(Cc2cc3cc(Cl)ccc3o2)c(=O)c2cscc12]

In certain embodiments, the compound of Formula (III) is

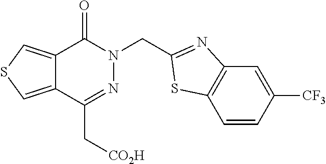 [SMILES: O=C(O)Cc1nn(Cc2nc3cc(C(F)(F)F)ccc3s2)c(=O)c2cscc12]

or a pharmaceutically acceptable salt thereof.

In certain embodiments, the compound of formula (III) is

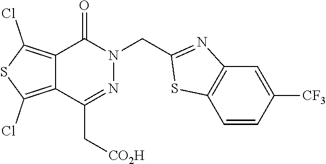 [SMILES: O=C(O)Cc1nn(Cc2nc3cc(C(F)(F)F)ccc3s2)c(=O)c2c(Cl)sc(Cl)c12]

or a pharmaceutically acceptable salt thereof.

The term “alkyl”, as used herein, unless otherwise indicated, refers to a monovalent aliphatic hydrocarbon radical having a straight chain, branched chain, monocyclic moiety, or polycyclic moiety or combinations thereof, wherein the radical is optionally substituted at one or more carbons of the straight chain, branched chain, monocyclic moiety, or polycyclic moiety or combinations thereof with one or more substituents at each carbon, where the one or more sub stituents are independently C₁-C₁₀ alkyl. Examples of “alkyl” groups include methyl, ethyl, propyl, isopropyl, butyl, iso-butyl, sec-butyl, tert-butyl, pentyl, hexyl, heptyl, cyclopropyl, cyclobutyl, cyclopentyl, cyclohexyl, cycloheptyl, norbornyl, and the like.

The term “halogen” or “halo-”, as used herein, means chlorine (Cl), fluorine (F), iodine (I) or bromine (Br).

As used herein, the term “acyl” is used in a broad sense to designate radicals of the type RCO—, in which R represents an organic radical which may be an alkyl, aralkyl, aryl, alicyclic or heterocyclic radical, substituted or unsubstituted, saturated or unsaturated; or, differently defined, the term “acyl” is used to designate broadly the monovalent radicals left when the OH group of the carboxylic radical is removed from the molecule of a carboxylic acid.

The term “alkoxy” is employed to designate a group of the formula: —O—R wherein R is an alkyl group, which optionally contains substituents, such as halogen. Preferably, the term “alkoxy” is employed to designate an alkoxy with an alkyl group of 1 to 6 carbon atoms. Most preferably, the term “alkoxy” is employed to designate an alkoxy with an alkyl group of 1 to 3 carbon atoms, such as methoxy or ethoxy.

The term “cycloalkyl group” is used herein to identify cycloalkyl groups having 3-6 carbon atoms preferably cyclopropyl, cyclobutyl, cyclopentyl and cyclohexyl.

The term “solvate” as used herein means a compound, or a pharmaceutically acceptable salt thereof, wherein molecules of a suitable solvent are incorporated in the crystal lattice. A suitable solvent is physiologically tolerable at the dosage administered. Examples of suitable solvents are ethanol, water and the like. When water is the solvent, the molecule is referred to as a “hydrate.”

A “prodrug” refers to an agent, which is converted into the parent drug in vivo. Prodrugs are often useful because, in some situations, they are easier to administer than the parent drug. They are bioavailable, for instance, by oral administration whereas the parent drug is either less bioavailable or not bioavailable. The prodrug also has improved solubility in pharmaceutical compositions over the parent drug. For example, the compound carries protective groups which are split off by hydrolysis in body fluids, e.g., in the bloodstream, thus releasing active compound or is oxidized or reduced in body fluids to release the compound. The term “prodrug” may apply to such functionalities as, for example; the acid functionalities of the compounds of formula I. Prodrugs may be comprised of structures wherein an acid group is masked, for example, as an ester or amide. Further examples of prodrugs are discussed herein. See also Alexander et al. (J. Med. Chem. 1988, 31, 318), which is incorporated by reference. Examples of prodrugs include, but are not limited to, derivatives and metabolites of a compound that include biohydrolyzable moieties such as biohydrolyzable amides, biohydrolyzable esters, biohydrolyzable carbamates, biohydrolyzable carbonates, and biohydrolyzable phosphate analogues. Prodrugs are also described in, for example, The Practice of Medicinal Chemistry (Camille Wermuth, ed., 1999, Academic Press; hereby incorporated by reference in its entirety). In certain embodiments, prodrugs of compounds with carboxyl functional groups are the lower alkyl esters of the carboxylic acid. The carboxylate esters are conveniently formed by esterifying any of the carboxylic acid moieties present on the molecule. Prodrugs can typically be prepared using well-known methods, such as those described by Burger's Medicinal Chemistry and Drug Discovery 6^{th} ed. (Donald J. Abraham ed., 2001, Wiley) and Design and Application of Prodrugs (H. Bundgaard ed., 1985, Harwood Academic Publishers Gmfh; each of which hereby incorporated by reference in its entirety). Biohydrolyzable moieties of a compound of Formula I (a) do not interfere with the biological activity of the compound but can confer upon that compound advantageous properties in vivo, such as uptake, duration of action, or onset of action; or (b) may be biologically inactive but are converted in vivo to the biologically active compound. Examples of biohydrolyzable esters include, but are not limited to, lower alkyl esters, alkoxyacyloxy esters, alkyl acylamino alkyl esters, and choline esters. Examples of biohydrolyzable amides include, but are not limited to, lower alkyl amides, a-amino acid amides, alkoxyacyl amides, and alkylaminoalkylcarbonyl amides. Examples of biohydrolyzable carbamates include, but are not limited to, lower alkylamines, substituted ethylenediamines, amino acids, hydroxyalkylamines, heterocyclic and heteroaromatic amines, and polyether amines.

The term “salt” includes salts derived from any suitable of organic and inorganic counter ions well known in the art and include, by way of example, hydrochloric acid salt or a hydrobromic acid salt or an alkaline or an acidic salt of the aforementioned amino acids. The term is intended to include salts derived from inorganic or organic acids including, for example hydrochloric, hydrobromic, sulfuric, nitric, perchloric, phosphoric, formic, acetic, lactic, maleic, fumaric, succinic, tartaric, glycolic, salicylic, citric, methanesulfonic, benzenesulfonic, benzoic, malonic, trifluoroacetic, trichloroacetic, naphthalene-2 sulfonic and other acids; and salts derived from inorganic or organic bases including, for example sodium, potassium, calcium, ammonium or tetrafluoroborate. Exemplary pharmaceutically acceptable salts are found, for example, in Berge, et al. (J. Pharm. Sci. 1977, 66(1), 1; and U.S. Pat. Nos. 6,570,013 and 4,939,140; each hereby incorporated by reference in its entirety). Pharmaceutically acceptable salts are also intended to encompass hemi-salts, wherein the ratio of compound: acid is respectively 2:1. Exemplary hemi-salts are those salts derived from acids comprising two carboxylic acid groups, such as malic acid, fumaric acid, maleic acid, succinic acid, tartaric acid, glutaric acid, oxalic acid, adipic acid and citric acid. Other exemplary hemi-salts are those salts derived from diprotic mineral acids such as sulfuric acid. Exemplary preferred hemi-salts include, but are not limited to, hemimaleate, hemifumarate, and hemisuccinate.

The term “acid” contemplates all pharmaceutically acceptable inorganic or organic acids. Inorganic acids include mineral acids such as hydrohalic acids, such as hydrobromic and hydrochloric acids, sulfuric acids, phosphoric acids and nitric acids. Organic acids include all pharmaceutically acceptable aliphatic, alicyclic and aromatic carboxylic acids, dicarboxylic acids, tricarboxylic acids, and fatty acids. Preferred acids are straight chain or branched, saturated or unsaturated C₁-C₂₀ aliphatic carboxylic acids, which are optionally substituted by halogen or by hydroxyl groups, or C₆-C₁₂ aromatic carboxylic acids. Examples of such acids are carbonic acid, formic acid, fumaric acid, acetic acid, propionic acid, isopropionic acid, valeric acid, alpha-hydroxy acids, such as glycolic acid and lactic acid, chloroacetic acid, benzoic acid, methane sulfonic acid, and salicylic acid. Examples of dicarboxylic acids include oxalic acid, malic acid, succinic acid, tartaric acid and maleic acid. An example of a tricarboxylic acid is citric acid. Fatty acids include all pharmaceutically acceptable saturated or unsaturated aliphatic or aromatic carboxylic acids having 4 to 24 carbon atoms. Examples include butyric acid, isobutyric acid, sec-butyric acid, lauric acid, palmitic acid, stearic acid, oleic acid, linoleic acid, linolenic acid, and phenylsteric acid. Other acids include gluconic acid, glycoheptonic acid and lactobionic acid.

### III. Compositions

The compounds can be administered in the form a suitable composition, such as a pharmaceutical composition. Pharmaceutical compositions are physiologically acceptable and typically include the active compound and a carrier. The term “carrier” refers to a diluent, adjuvant, excipient, or vehicle with which a compound is administered. Non-limiting examples of such pharmaceutical carriers include liquids, such as water, alcohols and oils, including those of petroleum, animal, vegetable or synthetic origin, such as peanut oil, soybean oil, mineral oil, sesame oil and the like. The pharmaceutical carriers may also be saline, gum acacia, gelatin, starch paste, talc, keratin, colloidal silica, urea, and the like. In addition, auxiliary, stabilizing, thickening, lubricating and coloring agents may be used. Suitable pharmaceutical carriers, dosage forms and formulation techniques to produce pharmaceutical compositions for a desired type of administration are well-known and are described, for example, in Remington's Pharmaceutical Sciences (Alfonso Gennaro ed., Krieger Publishing Company (1997); Remington's: The Science and Practice of Pharmacy, 21^{st} Ed. (Lippincot, Williams & Wilkins (2005); Modern Pharmaceutics, vol. 121 (Gilbert Banker and Christopher Rhodes, CRC Press (2002); each of which hereby incorporated by reference in its entirety). Suitable pharmaceutical carriers, dosage forms and formulation techniques for dermatological and cosmetic uses are also are well-known and are described, for example, in Handbook of Cosmetic Science and Technology, Fourth Edition, edited by Andre O. Barel, Marc Paye, Howard I. Maibach, CRC Press, 2014, the contents of which is hereby incorporated by reference in its entirety.

The composition can be in a desired form, such as a table, capsule, solution, emulsion, suspension, gel, sol, or colloid that is physiologically and/or pharmaceutically acceptable. The compositions can be prepared for administration by any suitable route such as ocular (including periocular and intravitreal administration), oral, parenteral (e.g., subcutaneous, intravenous, intra-arterial, intrathecal and intraperitoneal administration), intranasal, anal, vaginal, inhalation (e.g, as a powder or liquid aerosol) and topical administration. Oral or parenteral (e.g. intravenous) administration is generally preferred.

If desired, the pharmaceutical composition can include a buffer, for example with alkaline buffers, e.g., ammonium buffer, acidic buffers, e.g., ethanoates, citrates, lactates, acetates, etc., or zwitterionic buffers, such as, glycine, alanine, valine, leucine, isoleucine and phenylalanine, Kreb's-Ringer buffer, TRIS, IVIES, ADA, ACES, PIPES, MOPSO, cholamine chloride, MOPS, BES, TES, HEPES, DIPSO, MOBS, TAPSO, acetamidoglycine, TEA, POPSO, HEPPSO, EPS, HEPPS, Tricine, TRIZMA, Glycinamide, Glycyl-glycine, HEPBS, Bicine, TAPS, AMPB, CHES, AMP, AMPSO, CAPSO, CAPS, and CABS.

When the composition is in a liquid form, a carrier can be a solvent or dispersion medium comprising but not limited to, water, ethanol, polyol (e.g., glycerol, propylene glycol, liquid polyethylene glycol, etc.), lipids (e.g., triglycerides, vegetable oils, liposomes) and combinations thereof. The proper fluidity can be maintained, for example, by the use of a coating, such as lecithin; by the maintenance of the required particle size by dispersion in carriers such as, for example liquid polyol or lipids; by the use of surfactants such as, for example hydroxypropylcellulose; or combinations thereof such methods. If desired, tonicity adjusting agents can be included, such as, for example, sugars, sodium chloride or combinations thereof In some embodiments, the composition is isotonic.

The compositions may also include additional ingredients, such as acceptable surfactants, co-solvents, emollients, agents to adjust the pH and osmolarity and/or antioxidants to retard oxidation of one or more component.

Suitable carriers for oral administration are well-known and comprise inert diluents, edible carriers or combinations thereof. Examples of pharmaceutically acceptable carriers may include, for example, water or saline solution, polymers such as polyethylene glycol, carbohydrates and derivatives thereof, oils, fatty acids, or alcohols. Surfactants such as, for example, detergents, are also suitable for use in the formulations. Specific examples of surfactants include polyvinylpyrrolidone, polyvinyl alcohols, copolymers of vinyl acetate and of vinylpyrrolidone, polyethylene glycols, benzyl alcohol, mannitol, glycerol, sorbitol or polyoxyethylenated esters of sorbitan; lecithin or sodium carboxymethylcellulose; or acrylic derivatives, such as methacrylates and others, anionic surfactants, such as alkaline stearates, in particular sodium, potassium or ammonium stearate; calcium stearate or triethanolamine stearate; alkyl sulfates, in particular sodium lauryl sulfate and sodium cetyl sulfate; sodium dodecylbenzenesulphonate or sodium dioctyl sulphosuccinate; or fatty acids, in particular those derived from coconut oil, cationic surfactants, such as water-soluble quaternary ammonium salts of formula N R′R″R′″R″″Y″, in which the R radicals are identical or different optionally hydroxylated hydrocarbon radicals and Y″ is an anion of a strong acid, such as halide, sulfate and sulfonate anions; cetyltrimethylammonium bromide is one of the cationic surfactants which can be used, amine salts of formula NR′R′R″, in which the R radicals are identical or different optionally hydroxylated hydrocarbon radicals; octadecylamine hydrochloride is one of the cationic surfactants which can be used, non-ionic surfactants, such as optionally polyoxyethylenated esters of sorbitan, in particular Polysorbate 80, or polyoxyethylenated alkyl ethers; polyethylene glycol stearate, polyoxyethylenated derivatives of castor oil, polyglycerol esters, polyoxyethylenated fatty alcohols, polyoxyethylenated fatty acids or copolymers of ethylene oxide and of propylene oxide, amphoteric surfactants, such as substituted lauryl compounds of betaine.

If desired, an oral composition may comprise one or more binders, excipients, disintegration agents, lubricants, flavoring agents, and combinations thereof In certain embodiments, a composition may comprise one or more of the following: a binder, such as, for example, gum tragacanth, acacia, cornstarch, gelatin or combinations thereof; an excipient, such as, for example, dicalcium phosphate, mannitol, lactose, starch, magnesium stearate, sodium saccharine, cellulose, magnesium carbonate or combinations thereof; a disintegrating agent, such as, for example, corn starch, potato starch, alginic acid or combinations thereof a lubricant, such as, for example, magnesium stearate; a sweetening agent, such as, for example, sucrose, lactose, saccharin or combinations thereof; a flavoring agent, such as, for example peppermint, oil of wintergreen, cherry flavoring, orange flavoring, etc., or combinations thereof containing two or more of the foregoing.

Topical formulations for application to the skin typically include one or more of a vehicle or solvent, permeation enhancer, thickening or gelling agent, humectant, emulsifying or solubilizing agent, emollient, stiffening agent or ointment base. See, e.g., Chang et al., The AAPS Journal 15:1, 41-52, 2012. DOI:10.10.1208/s12248-012-9411-0). Suitable vehicles or solvents include, purified water, Hexylene glycol, Propylene glycol, Oleyl alcohol, Propylene carbonate, Mineral oil, and the like. Suitable permeation enhancers include Propylene glycol, Ethanol, Isopropyl Alcohol, Oleic acid, Polyethylene glycol, and the like. Suitable thickening or gelling agents include Carbomer, Methyl cellulose, Sodium carboxyl methyl cellulose, Carrageenan, Colloidal silicon dioxide, Guar gum, Hydroxypropyl cellulose, Hydroxypropyl methyl cellulose, Gelatin, Polyethylene oxide, Alginic acid, Sodium alginate, Fumed silica, and the like. Suitable humectants include Glycerin, Propylene glycol, Polyethylene glycol, Sorbitol solution, 1,2,6 Hexanetriol, and the like. Suitable emulsifying or solubilizing agents include Polysorbate 20, Polysorbate 80, Polysorbate 60, Poloxamer, Emulsifying wax, Sorbitan monostearate, Sorbitan monooleate, Sodium lauryl sulfate, Propylene glycol monostearate, Diethylene glycol monoethyl ether, Docusate sodium, and the like. Suitable emollients, stiffening agents, ointment bases include Carnauba wax, Cetyl alcohol, Cetyl ester wax, Emulsifying wax, Hydrous lanolin, Lanolin, Lanolin alcohols, Microcrystalline wax, Paraffin, Petrolatum, Polyethylene glycol, Stearic acid, Stearyl alcohol, White wax, Yellow wax, and the like. If desired, preservatives, anti-oxidants, chelating agents, acidifying, alkalizing or buffering agents can also be included in topical compositions.

Typical pharmaceutically acceptable compositions can contain a an AR inhibitor and/or a pharmaceutically acceptable salt thereof at a concentration ranging from about 0.01 to about 20 wt %, such as 0.01 to about 15 wt %, 0.01 to about 10 wt %, or about 0.01 to about 5 wt %. The compositions for parenteral administration or typically sterile.

### IV. Combination Therapy

The methods described herein include the administration of an AR inhibitor and one more additional therapeutic agent. The additional therapeutic agents may be administered before, concurrently with or after the AR inhibitor, but in a manner that provides for overlap of the pharmacological activity of the AR inhibitor and the additional therapeutic agent.

The additional therapeutic agent can be, for example, second aldose reductase inhibitor, an antioxidant, an antiviral, an anti-inflammatory or any combination of the foregoing. For example, the second aldose reductase can zopolrestat, epalrestat, ranirestat, berberine and sorbinil, as described in, e.g., U.S. Pat. Nos. 4,939,140; 6,159,976; and 6,570,013. Preferably, the second aldose reductase inhibitor is selected from ponalrestat, epalrestat, sorbinil or sorbinol, imirestat, AND-138, CT-112, zopolrestat, zenarestat, BAL-AR18, AD-5467, M-79175, tolrestat, alconil, statil, berberine or SPR-210.

Other therapeutic agents that can be administered with an AR inhibitor include, for example, antioxidants. Suitable antioxidants include vitamins, such as vitamin C (L-ascorbic acid), vitamin B3 (niacinamide) and vitamin E (alpha-tocopheraol), polyphenols (e.g., from green tea) and flavonoids (e.g. from soya). Suitable antivirals and anti-inflammatories include, for example, remdesivir, anti-IL6 agents (e.g. tocilizumab, sarilumab, siltuximab, olokizumab, elsilimomab, sirulumag, levilimab).

### V. Example

Treatment of Critical COVID-19

A patient is diagnosed with critical COVID-19 and shows signs of ARDS and/or ALI. The patient is admitted to the critical care unit and treated with Compound A at a dose of 1500 mg oral twice a day by the attending critical care physician. Treatment with Compound A continues, signs of ARDS and/or ALI are diminished and viral burden is reduced. The patient recovers from COVID-19 and is discharged from the hospital.

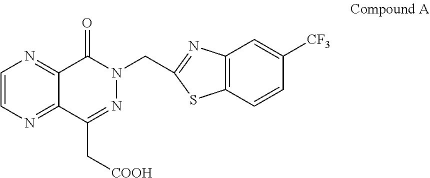 [SMILES: O=C(O)Cc1nn(Cc2nc3cc(C(F)(F)F)ccc3s2)c(=O)c2nccnc12]

An Open Label Study of Compound A in Hospitalized Patients with COVID-19

An open-label prospective phase 2 clinical of Compound A (caficrestat) treatment in high risk patients hospitalized with COVID-19. The high risk patients had COVID-19 infection and history of diabetes mellitus and heart disease. The study design prospectively included selection of matched controls from a contemporaneous registry of COVID-19 patients hospitalized at the same institution.

Aldose Reductase, the rate-limiting step of the polyol metabolic pathway, plays a critical role in mediation of oxidative tissue damage in setting of inflammation induced by infection or ischemia and may contribute to NLRP3 inflammasome activation in diabetic patients with COVID-19. (Ruef J, et al., Arterioscler Thromb Vasc Biol. 2000; 20:1745-52) Compound A (caficrestat) is a selective inhibitor of aldose reductase enzymatic activity that decreases biomarkers of myocardial stress and is currently being studied in a Phase 3 global registrational study in patients with diabetic cardiomyopathy (ARISE-HF). Aldose reductase inhibition with Compound A (caficrestat) might represent a novel therapeutic approach to reduce NLRP3 inflammasome activation and risk of adverse outcomes, including development of ARDS, ALI and/or cardomyopathy in high risk with COVID-19. Aldose reductase is inactive in a “healthy” state, but becomes activated under oxidative stress causing inflammatory damage, such as ARDS resulting in acute lung inflammation and damage in COVID-19.

Adults with diabetes mellitus, and history of hypertension, coronary artery disease, or heart failure, and COVID-19 infection requiring hospitalization at New York University Langone Health (NYULH) Hospitals were eligible for enrollment in the prospective open-label clinical trial. Diabetes mellitus was defined as history of diabetes mellitus documented in the medical record or blood glucose level >126 mg/dl during the index hospitalization. COVID-19 infection was confirmed by laboratory RT-PCR testing. Key exclusion criteria included inability to take study drug by mouth or nasogastric tube, participation in another FDA-regulated investigational drug placebo-controlled clinical trial within previous 30 days, and women of childbearing potential. The study protocol was approved by the institutional review board of New York University Grossman School of Medicine. All participants provided written informed consent prior to initiation of study procedures. The study was registered as a clinical trial on the clinicaltrials.gov website (Identifier NCT04365699) prior to the start of enrollment.

Compound A (caficrestat) 1500 mg twice daily was administered by mouth or nasogastric tube for up to 14 days. The electronic medical record was used to extract information on demographics, co-morbid medical conditions, COVID-19 complications, adverse events, hospital discharge and transfer information. The World Health Organization COVID-19 ordinal scale for clinical improvement status was determined at 30 days after the last dose of study drug. The study design prospectively planned analysis of matched control subjects of patients hospitalized with COVID-19 infections at the study enrollment sites. Clinical data related to an index hospitalization for COVID-19 infection for matched control subjects were extracted from a deidentified data registry.

Compound A (caficrestat) 500mg capsules were provided by the sponsor (Applied Therapeutics Inc., New York N.Y.), and stored and distributed by the NYULH Investigational Pharmacy. A dose of AT-001 (caficrestat) 1500 mg (3 capsules) was administered to the study subjects by mouth twice daily for up to 14 days per discretion of the investigators and treatment team. If hospital discharge occurred prior to completion of treatment, the remaining supply of AT-001 (caficrestat) capsules were given to the participant at the time of discharge with instructions for self-administration post-discharge. Study drug administration was tracked in the electronic medical record prior to discharge, and by daily telephone contact post-discharge.

To provide observational control data in hospitalized COVID-19 patients not treated with Compound A (caficrestat), matched controls from a contemporaneous de-identified registry of COVID-19 patients hospitalized at the same institution were selected according to two matching strategies based on ICD-10 billing codes and clinical characteristics. As of Feb. 15, 2020, the NYULH registry database had captured data on approximately 43,000 patients with COVID-19 diagnosis hospitalized in the NYULH Tisch and NYULH Long Island Hospitals (11,563 patients with at least one inpatient encounter diagnosis for diabetes mellitus, and 3608 patients with at least one inpatient encounter diagnosis for hypertension). There were 1931 patients with history of diabetes mellitus, 559 patients with history of hypertension, and 190 patients with history of both diabetes mellitus and hypertension who also had available data to match participants who received Compound A (caficrestat). The first matching approach selected all subjects in the registry with diabetes mellitus and hypertension, and available data to match participants who received Compound A (caficrestat) for gender, age group (in bins of 5 years), weight, and C-reactive protein (CRP) value at the time of hospital admission. The second matching approach selected all subjects in the registry with diabetes mellitus and available data to match participants who received Compound A (caficrestat) for gender, age group (in bins of 5 years), and weight ±0.5 kgs interval. Each participant in the prospective Compound A (caficrestat) study had more than one matching control group patient. There was no specific hierarchy of selection for the matches, as the variables were considered as independent of each other. Descriptive statistics are reported for the participant prospectively collected data and retrospective data from the matched cohorts. The median length of stay with its 95% confidence interval for participants who received Compound A (caficrestat) and matched control subjects and the between group difference in median of length of stay with its 95% confidence interval (using the Hodges-Lehmann estimate of location shift) for the Compound A (caficrestat) and the control groups are reported.

Ten patients hospitalized at NYULH Tisch and NYULH Long Island hospitals for COVID19 infection were enrolled in the prospective trial and received open-label treatment with Compound A (caficrestat). All participants in the prospective study had history of diabetes mellitus and hypertension, and were treated with supplemental oxygen. Based on the two sets of matching control criteria, 16 and 60 patients were identified for matching control groups 1 and 2, respectively. Table 1 summarizes demographic and clinical characteristics of the prospective clinical trial participants and two control groups.

TABLE 1

| | All patients | | |
|---|---|---|---|
| | | Control | Control |
| | Compound A | group 1 | group 2 |
| | (N = 10) | (N = 16) | (N = 60) |
| Age (years) | 66.4 ± 6.6 | 65 ± 7.7 | 65.1 ± 5.6 |
| Male sex (%) | 80 | 87.5 | 80 |
| Weight (kg) | 86.8 ± 16.2 | 93.3 ± 9.5 | 87.7 ± 13.7 |
| C-Reactive | 52.5 (7.7; 176.5) | 79.2 (8.3; 183) | 52.9 (6.6; 108.9) |
| Protein | | | |
| (mg/dl)* | | | |

Five of the 10 participants completed >80% of scheduled 28 doses of Compound A (caficrestat), with overall mean of 15 completed doses. Compound A (caficrestat) treatment was discontinued early due to adverse events not related to study drug in two participants, due to mild-moderate grade adverse events possibly linked to study drug in three participants (nausea/GI upset (n=2) and localized skin rash (n=1)), and due to patient preference in three participants. Safety laboratory monitoring during hospitalization did not demonstrate any treatment-related abnormal findings.

Of the 10 participants treated with Compound A (caficrestat) in the prospective clinical trial, eight survived, and were discharged from the hospital, with median hospital length of stay of 5 days (range 3-35 days), and two died during the index hospitalization due to COVID-19 complications (progressive hypoxic respiratory failure). The percent mortality observed in the Compound A (caficrestat) group (20%) was numerically lower than that observed in both matched controls groups (first matched control group 5 out of 16 subjects (31.3%) and second matched control group 17 out of 60 subjects (28.3%)). Length of hospital stay observed in the Compound A (caficrestat) group was numerically less than length of hospital stay in the two matched control groups (Table 2).

TABLE 2

| | All Subjects | | | | | Surviving Subjects | | |
|---|---|---|---|---|---|---|---|---|
| | Compound A | Match 1 | | Match 2 | | Compound A | Match 1 | Match 2 |
| N | 10 | 16 | | 60 | | 8 | 11 | 43 |
| Median LOS | 5 (4, 29) | 10 | (5, 20) | 24.5 | (16, 36) | 5 (4, 35) | 6 (4, 85) | 16 (10, 36) |
| (95% CI) | | | | | | | | |
| Estimated | — | 1.5 | (−2, 12) | 14 | (1, 37) | — | 1 (−2, 19) | 9 (0, 42) |
| difference in | | | | | | | | |
| median LOS | | | | | | | | |

Previous observational studies have reported that patients with hypertension, heart failure, diabetes mellitus, and obesity are at greater risk of severe disease complications and death due to COVID-19. The underlying causal pathways linking cardiometabolic diseases to adverse outcomes in COVID-19 remain uncharacterized, but may be in part be attributable to upregulation of the NLRP3 inflammasome pathway and induction of trained innate immunity in these populations. In this pilot study of COVID-19 patients with these comorbid conditions, treatment with the selective, potent aldose reductase inhibitor Compound A (caficrestat) was associated with numerically shorter length of hospital stay and decreased mortality when compared with contemporary matched control subjects treated for COVID-19 at the same institution. Compound A (caficrestat) was well tolerated, consistently with a favorable tolerability profile observed in prior studies of non-COVID-19 patients with diabetic cardiomyopathy. In contrast to other therapies currently employed for treatment of COVID-19, such as remdesivir, monoclonal antibodies and convalescent plasma, Compound A (caficrestat) is dosed orally and can be administered in an outpatient setting.

Despite important advances in prevention and treatment, the COVID-19 pandemic continues to be a major global health crisis, and strategies to mitigate risk of acute disease complications and death are urgently needed. COVID-19 is associated with increased risk of adverse outcomes in patients with diabetes mellitus, and also may lead to acute and chronic alterations in glycemic control. Although strategies for disease eradication through vaccination have been at the forefront of public health strategies, management of the acute and long-term complications of the disease in affected individuals remains an important consideration that may persist beyond the duration of the current COVID-19 pandemic.

This study assessed the safety, tolerability and efficacy of aldose reductase inhibition with Compound A (caficrestat) in ten hospitalized patients with COVID-19 and co-morbid diabetes mellitus and hypertension. Compound A (caficrestat) was generally safe and well tolerated. Analysis of mortality and length of hospital stay showed that both percent mortality and length of hospital stay were numerically lower in patients treated with Compound A (caficrestat) in comparison to matched controls.

Mortality in COVID-19 patients without co-morbidity risk factors who were admitted to New York hospitals was approximately 10-20%. (JAMA. 2020:323(20)2052-2059. Doi:10.1001/jama.2020.6775; N Engl J Med 2020; 382:2372-2374, doi:10.1056/NEJMc2010419) In contrast, mortality in COVID-19 patients with any single risk factor (e.g., diabetes, hypertension, coronary artery disease) is increased to 30-40%, more than double that the no risk factor patients. (Id.) In this study, a small cohort of high-risk patients with three risk factors (diabetes, hypertension and coronary artery disease) were treated with Compound A (caficrestat), and mortality was decreased to the level observed for the non-high risk population. Eight of the ten treated patients survived, were discharged from the hospital and were healthy at follow up 30 days post discharge. None of the treated patients progressed to mechanical ventilation.

Treatment of Critical COVID-19 Patients with ARDS

Five critical COVID-19 patients admitted to the intensive care unit with severe ARDS were treated with Compound A (caficrestat) 1500 mg twice a day. All patients were on 100% high flow oxygen, had failed all other available treatment options, including anti-IL⁶/anti-IL-17 therapy, and were at high risk of rapid progression to mechanical ventilation. The patients were treated under Emergency Investigational New Drug Applications as the last option to prevent mortality. Four of the five treated patients recovered and were discharged from the hospital. One patient, who had a do not resuscitate order in place, died following progression to mechanical ventilation.

Unless otherwise defined, all technical and scientific terms used herein have the same meaning as commonly understood by one of ordinary skill in the art to which this disclosure belongs.

Various aspects of the invention have been described in this disclosure. Such aspects may, however, be embodied in many different forms and should not be construed as limited to the particular embodiments set forth herein; rather, these embodiments are provided so that this disclosure will be thorough and complete.

Although methods and materials similar or equivalent to those described herein can be used in the practice or testing of the present disclosure, suitable methods and materials are described in the foregoing paragraphs. In addition, the materials, methods, and examples are illustrative only and not intended to be limiting. All United States patents and published or unpublished United States patent applications cited herein are incorporated by reference. All published foreign patents and patent applications cited herein are hereby incorporated by reference. All published references, documents, manuscripts, scientific literature cited herein are hereby incorporated by reference.

1. (canceled)

2. A method for treating acute respiratory distress syndrome (ARDS), acute lung inflammation (ALI), or acute lung injury, comprising administering to a subject in need thereof a therapeutically effective amount of an aldose reductase inhibitor (ARI).

3-6. (canceled)

7. The method of claim 2, wherein the subject has an infection, and the ARDS, ALI, or acute lung injury is related to or caused by the infection.

8. A method for treating cardiac injury related to or caused by an infection, comprising administering to a subject in need thereof a therapeutically effective amount of an aldose reductase inhibitor (ARI).

9. (canceled)

10. A method for treating an infection, decreasing pathogen burden in a subject with an infection, or inhibiting pathogen replication in a subject with an infection, comprising administering to a subject in need thereof a therapeutically effective amount of an aldose reductase inhibitor (ARI).

11-19. (canceled)

20. The method of claim 7, wherein the infection is a viral infection

21. The method of claim 20, wherein the viral infection is a respiratory virus or a herpes virus.

22. (canceled)

23. The method of claim 21, wherein the respiratory virus is a respiratory syncytial virus (RSV), rhinovirus, parainfluenza virus (PIV), adenovirus, metapneumovirus, enterovirus, bocavirus, influenza virus, or coronavirus.

24-25. (canceled)

26. The method of claim 23, wherein the coronavirus is SARS-CoV-1, SARS-CoV-2, or MERS-CoV.

27. (canceled)

28. The method of claim 21, wherein the herpesvirus is herpes simplex virus or cytomegalovirus.

29. The method of claim 7, wherein the infection is a bacterial infection, a fungal infection, or a parasitic infection.

30. The method of claim 29, wherein the bacterial infection is selected from the group consisting of Streptococcus pneumoniae, Group B streptococci, Group A streptococci, Staphylococcus aureus, Hemophilus influenzae, Nisseria sp., Enterococcus sp., Mycoplasma pneumoniae, Chlamydia pneumoniae, Mycobacterium tuberculosis, and Mycobacterium avium.

31. (canceled)

32. The method of claim 29, wherein the fungal infection is selected from Aspergillus sp., Blastomyces sp., and Cryptococcus sp.

33. (canceled)

34. The method of claim 29, wherein the parasitic infection is selected from Pneumocystis jiroveci, Plasmodium falciparum, or Plasmodium vivax.

35. The method of claim 7, wherein the subject has sepsis.

36. The method of claim 8, wherein the subject has infection-related or infection-caused cardiomyopathy, myocarditis, or acute cardiac injury.

37. The method of claim 8, wherein the subject has underlying cardiomyopathy.

38-39. (canceled)

40. The method of claim 36, wherein the acute cardiac injury is characterized by elevated troponin levels.

41. The method of claim 8, wherein the subject has elevated troponin levels, NTproBNP levels, and/or creatine kinase levels.

42-46. (canceled)

47. The method of claim 2, wherein the subject has influenza, Severe Acute Respiratory Syndrome (SARS), Middle East Respiratory Syndrome (MERS), or COVID-19.

48-49. (canceled)

50. The method of claim 2, wherein the ARI is a compound of any one of Formulas I-III, zopolrestat, epalrestat, or a salt of any of the foregoing.

51-52. (canceled)

53. The method of claim 2, wherein the ARI is 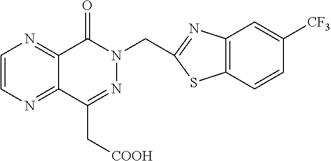 [SMILES: O=C(O)Cc1nn(Cc2nc3cc(C(F)(F)F)ccc3s2)c(=O)c2nccnc12]
or a salt thereof.

54. (canceled)
